(19) United States
(12) Patent Application Publication
(10) Pub. No.: US 2010/0055052 A1
(43) Pub. Date: Mar. 4, 2010

(54) Processing System for Oral Care Compositions

(76) Inventors: James Albert Berta, West Chester, OH (US); David Scott Howe, Hamilton, OH (US); David Michael Farren, Mason, OH (US); Steven Paul Miller, Cincinnati, OH (US); Shane Michael de la Harpe, Woking (GB); Volker Bernhard Deister, Gau-Algesheim (DE); Jeff Timothy Cullinane, Cincinnati, OH (US)

Correspondence Address: James Albert Berta, 8322 Taffy Drive, West Chester, OH, 45069, US

(21) Appl. No.: 12/198,255

(22) Filed: Aug. 26, 2008

(51) Int. Cl. A61K 8/18; A61Q 11/00

(52) U.S. Cl. 424/49

(57) ABSTRACT

This invention relates to a low loss liquid processing system used for manufacturing personal care and cleaning compositions, in particular oral care compositions such as dentifrice, mouthrinse and denture care products. The present process addresses manufacturing flexibility issues involved in making products to demand and the ability to proactively monitor intermediate compositions for quality assurance purposes. Specifically, the present manufacturing process provides faster variant turnaround times, shorter clean-up times between variants, significant material waste reduction and smaller effluent volumes than those of typical batch systems. As well, a process has been developed for more efficient addition of materials which are dissimilar in chemical composition and/or physical parameters to the original base material or of materials which may be temperature and shear sensitive.

### FIELD OF INVENTION

This invention relates to a processing system and use of the system to manufacture a wide variety of personal care and cleaning products, in particular products for oral care.

### BACKGROUND OF THE INVENTION

The health and appearance of teeth and the oral cavity are among the most important concerns of daily living. Consumers have various needs including cleaning and prevention of oral infection, dental plaque and calculus as well as stain removal, stain control, tooth whitening, control of bad breath and breath freshening. Thus, for oral care products for daily use such as dentifrice, rinses, and dental floss to provide thorough cleaning and care of the oral cavity, it is necessary to add multiple ingredients working by different mechanisms to provide the aforementioned benefits. Formulating such products presents many difficulties such as would arise from incompatibility of multiple ingredients. In addition, many different versions of products are required to take into account the differing needs and preferences of consumers, for example, with regard to flavor, appearance, texture and mouthfeel characteristics. Therefore there is a continuing need to improve production capabilities in order to provide a wide variety of oral care products to consumers.

Oral care compositions have traditionally been prepared using batch processes. In these processes, base materials or ingredients are separately prepared. These base materials are then combined in a mixing tank or reactor typically with continuous agitation and sometimes with application of additional mechanical and/or thermal energy. Additional base, finishing and/or reblend materials are separately prepared and are then added to the combined base materials. Often in batch processes, several steps and time delays would be necessary in order to add these additional materials which define the variations of the oral care compositions. This was often the case when such additional materials are dissimilar in chemical composition and/or physical parameters to the original base materials or when the additional materials are temperature and/or shear sensitive.

When a variation of an oral care composition is manufactured in a batch process, the mixing tank and most of its feed lines must be cleaned. Turnaround preparation is costly in both time and materials. It is particularly disadvantageous when the laborious turnaround is meant only to produce relatively slight variants of the first composition, for example, changing aesthetic components such as flavor, color or coolants.

Thus, a system was sought which would overcome the foregoing problems. More particularly, a system for manufacturing a wide variety of oral care products was developed which requires less capital with respect to mixing tanks, pumps and piping and provides significant cost savings resulting from minimizing material loss/waste and faster manufacturing.

### BRIEF DESCRIPTION OF THE DRAWING

FIG. 1 is a flow diagram of an embodiment of the present low loss liquid processing system used for manufacturing an oral care composition such as a dentifrice.

### SUMMARY OF THE INVENTION

This invention relates to a low loss liquid processing system used for manufacturing personal care and cleaning compositions, in particular oral care compositions such as dentifrice, mouthrinse and denture care products. The present process addresses manufacturing flexibility issues involved in making products to demand and the ability to proactively monitor intermediate compositions for quality assurance purposes. Specifically, the present manufacturing process provides faster variant turnaround times, shorter clean-up times between variants, significant material waste reduction and smaller effluent volumes than those of typical batch systems. As well, a process has been developed for more efficient addition of materials which are dissimilar in chemical composition and/or physical parameters to the original base material or of materials which may be temperature and shear sensitive.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at about 25° C., i.e., room temperature conditions, unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including dentifrice, toothpaste, tooth gel, subgingival gel, mouthrinse, mousse, foam, denture product, mouthspray, lozenge, chewable tablet or chewing gum. The oral care composition may also be incorporated onto strips or films for direct application or attachment to oral surfaces.

The term “dentifrice”, as used herein, includes paste, gel, liquid, powder or tablet formulations unless otherwise specified. The dentifrice composition may be a single phase composition or may be multiphase, i.e., a combination of two or more separate dentifrice compositions. The dentifrice composition may be in any desired form, such as deep striped, surface striped, multi-phase, multilayered, having a gel surrounding a paste, or any combination thereof Each dentifrice composition in a dentifrice comprising two or more separate dentifrice compositions may be contained in a physically separated compartment of a dispenser and dispensed side-by-side.

As used herein, the term “phase” as used herein refers to a homogeneous, physically distinct, and mechanically separable portion of matter present in a non-homogeneous physical-chemical system. Phases may be materials considered an intermediate and or a finished product. In one aspect, the phases herein are compositions with different colors. In one aspect, the phases comprise the same chemical compositions but with different colorants.

As used herein, the term “multiphase” or “multi-phase”, is meant that the phases of the present compositions occupy separate but distinct physical spaces inside the container or package in which they are stored, and may be in direct contact with one another but are not emulsified or mixed to any significant degree or the phases may be separated by a barrier. In one preferred embodiment of the present invention, the “multi-phase” oral care compositions comprise at least two visually distinct phases which are present within the container as a visually distinct pattern and/or displayed upon being dispensed. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. In one aspect, the phases may be various different colors, and/or include particles, glitter or pearlescent agents in at least one of the phases in order to offset its appearance from the other phase(s) present.

The term “dispenser”, as used herein, means any pump, tube, or container suitable for dispensing compositions such as dentifrices.

The term “teeth”, as used herein, refers to natural teeth as well as artificial teeth or dental prosthesis.

The term “orally-acceptable carrier” refer to safe and effective materials and conventional additives used in oral care compositions including but not limited to one or more of fluoride ion sources, anti-calculus or anti-tartar agents, buffers, abrasives such as silica, alkali metal bicarbonate salts, thickening materials, humectants, water, surfactants, titanium dioxide, flavor system, sweetening agents, xylitol, and coloring agents.

Active and other ingredients useful herein may be categorized or described by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

As used herein, a base material is a material that is employed as a sub-formulation and/or intermediate. As used herein, a sub-formulation may be a single raw material. The numerical modifier before the term “base material” does not necessarily imply a difference in chemical composition or physical parameters. For example, a first base material can have the same composition as a third base material.

As used herein, the term “personal care and cleaning compositions” include, unless otherwise indicated, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents; light duty dishwashing agents, especially those of the high-foaming type; machine dishwashing agents, including the various tablet, granular, liquid and rinse-aid types for household and institutional use; liquid cleaning, deodorizing and disinfecting agents, including antibacterial hand-wash types; laundry bars; soap bars; air and fabric deodorizers; car or carpet shampoos, bathroom cleaners; hair shampoos; hair-rinses; face wash; skin cleansers; shower gels; body washes; personal cleansing compositions; foam baths; metal cleaners; as well as, cleaning auxiliaries such as fabric enhancers, bleach additives and “stain-stick” or pre-treat types.

As used herein, combining refers to adding materials together with or without substantial mixing towards achieving homogeneity.

As used herein, mixing and blending interchangeably refer to combining and further achieving a relatively greater degree of homogeneity thereafter.

Traditional batch processes for manufacturing oral care products such as dentifrices in various forms, suffer from many disadvantages, in particular, significant ingredient(s) and finished product waste/loss, water consumption, landfill impacts and production time in order to deliver the large variety of formulations to market. Manufacturing a large variety of formulations increases the number of changeovers at the production sites, which increases the amount of time spent changing over, the amount of water used cleaning out mixing tanks, pipes and other equipment, and the amount of ingredients and product such as toothpaste washed down the drain. In an environment of diminishing resources these conditions are not sustainable, and are in need of a breakthrough.

The present process significantly eliminates the losses described above. In one embodiment, the present process changes dentifrice or toothpaste making from a batch system to a semi-continuous batch system, i.e., comprising multiple ingredient batches or streams. The present semi-continuous process is also useful for manufacturing other liquid or fluid products which may have a high solids content such as cleansing or detergent products containing solid ingredients such as abrasives. The present process is based on consolidation of core materials into one or more base formulations and separating from such base formulations the additional ingredients to produce variants that are meaningful to consumers. Such variant ingredients may include active as well as aesthetic agents. In one embodiment, the present process includes a liquids injection system which allows introduction of variant ingredients at the latest possible stage in processing. The liquids injection system features simultaneous addition of two or more ingredient streams into the main mixing step to produce the final product, thus further simplifying and shortening the manufacturing process. Product variants are produced by replacing one or more of the ingredient streams. These formula adjustments and injection technology allow consumer driven product differentiation while significantly reducing product scrap, capital expenditures, equipment downtime, and site water consumption for cleanup. This semi-continuous batch process also reduces the minimum run time for the packing lines, which in turn greatly reduces the finished product inventory. In traditional batch manufacturing processes, up to about 10% loss is typical, i.e., in wasted product during changeovers along with significant consumption of water for cleaning out the mixing and storage tanks. The present semi-batch process allows the elimination of up to 95% of the changeovers during formula making and intermediate storage portions of the process such that product loss is reduced to 3% or less and water savings up to 95% are achieved. The different components or streams of the present process are summarized in Table 1 below.

TABLE 1
Manufacturing Process Streams

| | Base | Organic | Aqueous | Non-Aqueous | Finished |
|---|---|---|---|---|---|
| | Stream(s) | Stream(s) | Stream(s) | Stream(s) | Product |
| Description | Partial | Primarily | Solutions of | Fluid stream | Combination |
| | formulation | organic | water-soluble | containing no | of base |
| | comprising | liquids, e.g., | or dispersible | more than 5% | stream(s) and |
| | core | flavors and | components | water | 1 or more |
| | ingredients, | perfumes | typically 50% | | other |
| | e.g., abrasive | | or more | | stream(s) |
| | | | water. | | |
| Viscosity (@ | 1 Pa · s-1000 Pa · s | 0.7 cP-1.2 cP | 0.7 cP-1000 cP | 0.5 Pa · s-500 Pa · s | 10 Pa · s-500 Pa · s |
| 1 sec⁻¹) | | | | | |
| % of Finished | 40%-99% | <3% | <30% | <10% | 100% |
| Product | | | | | |
| Density | 1.0 g/ml-1.6 g/ml | 0.7 g/ml-1.1 g/ml | 1.0 g/ml-1.4 g/ml | 1.2 g/ml-1.6 g/ml | 1.0 g/ml-1.6 g/ml |
| Flow Rate/ | >1.5 Tons/Hr | <10 l/m | <40 l/m | <20 l/m | 30 l/m-100 l/m |
| Production | | | | | |
| Rate | | | | | |
| Temperature | 15° C.-95° C. | 10° C.-80° C. | 15° C.-95° C. | 15° C.-95° C. | 15° C.-95° C. |

The process first requires preparation of one or more base or core formulation stream(s) which may be transferred into a separate tank for storage. The storage step in the process provides flexibility in manufacture location in that the base formulation(s) or any partial formulation can be stored and transported to another location where additional processing steps can occur. The storage step allows to be analyzed for quality assurance, allows the end product to be supplied to consumer demand and allows the manufacture of the end product to be in multiple locations, as well as remote locations. As used herein, the “storage,” “store,” or “storing” of a stream, partial formulation or finished product can occur for at a minimum of 5 minutes to an indefinite period which may last days, weeks, months or years.

The base stream(s) will typically make up the bulk of the formulation, from at least about 40% up to about 99% of the total formulation and will likely be a non-Newtonian fluid, i.e., having viscoelastic or shear-thinning properties. Additional ingredient streams to combine with the base stream(s) are then prepared. As with the base stream(s), these additional streams may be transferred into separate storage tanks. These streams may include one or more of predominantly aqueous stream(s) containing >50% water, essentially non-aqueous stream(s) containing less than 5% water and organic stream(s) comprising for example liquids such as flavor oils and organic solvents.

Downstream from storage, the one or more base stream(s) are discharged into a confluence or mixing region in conjunction with one or more of the other material streams for blending to result in a particular product variant. Blending of these materials occurs in a continuous flow manner. The process is intended to protect temperature and shear sensitive additives, such as flavors, fragrances and organic colorants which can be discharged at possibly a lower temperature and agitation than the other streams.

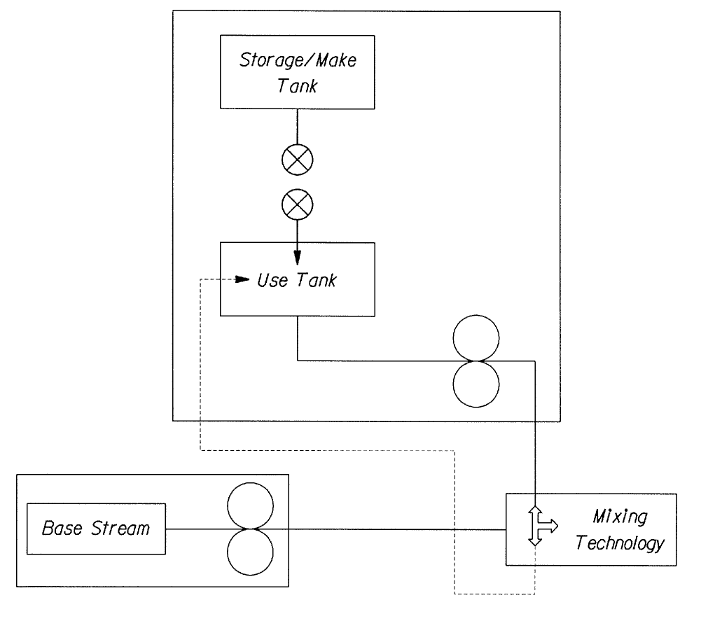

FIG. 1 shows a flow diagram of an embodiment of the present manufacturing process, wherein at least two ingredient streams, a base stream and an additive stream, are combined and blended. Once blended, the ingredient streams may constitute a personal care product such as a toothpaste or a cleaning composition such as a liquid body wash.

The process includes one or more additive systems (1) for preparing one or more ingredient streams to be mixed with one or more base streams (5) to form a product. The base and additive streams are each prepared in a system comprising a storage/make tank, a use tank and a dosing or metering pump (3), preferably servo controlled. The tanks may have a hard connection (2) or connected as needed. The storage/make tank is used to homogeneously produce and store an intermediate or partial formulation. A separate make tank and storage tank may also be used. The use tank acts as a reservoir to limit process interruptions. The dosing or metering pump (3) is used to accurately dose a fluid for injection into the mixing or blending region. An optional recycle loop (4) may be present to return a stream back to the use tank when needed.

The mixing technology may be any number of mixing devices including but not limited to static mixer, dynamic mixer, rotor stator, pin mill, extruder. The mixer produces the finished product which can then go to the filling line (7) which may comprise equipment for dosing finished product into a final package. The mixing device may include a confluence region (6) and mixing may begin where the streams initially come into contact at the confluence region, downstream thereof within the mixing device, or in both locations. Such confluence region will comprise two or more inlets (not shown) having inlet discharges (not shown) through which base stream(s) and other ingredient stream(s) are supplied. Such inlet discharges may be spaced throughout the confluence region in any manner. For example, such inlet discharges may be in close proximity to each other or widely spaced apart and they may lie in a common plane or different planes. Thus, such inlet discharges may be equally or unequally spaced circumferentially, radially, and/or longitudinally. Further, the inlet discharges may have equal or unequal cross sectional areas, shapes, lengths and flow rates therethrough. In one aspect, the inlet discharges may be closely juxtaposed with an inline mixer, so that mixing of the materials occurs almost immediately in the confluence region. In a preferred embodiment the various streams are simultaneously injected into the final mixing device to produce the finished product. The confluence region may further comprise at least one common outlet (not shown) for discharging the materials that have been supplied to the confluence region. At least one common outlet for discharging the materials may be designed such that the discharged matter flows into the mixing region.

After mixing all the material streams, the final product can leave the mixing region through at least one common outlet to the filling line (7) and be supplied for example, into a single container or plural containers (not shown) having equal or unequal volumes. The container(s) may be insertable into and removable from the system. The container(s) containing the product may be ultimately shipped and sold to the consumer, or may be used for transport and storage of the mixture as an intermediate. Thus, the container(s) may be selected from a bulk storage device, for example, a tank, a tank car, or rail car, or a final package, for example, a tube, bottle and/or a tottle. The container(s) may be provided with a frangible or resealable closure as are well known in the art, and be made of any material suitable for containing the materials combined according to the present invention.

In one aspect, the material streams are supplied to the confluence/mixing region by one or more inlet tube(s) inserted therein. The flow can be directed radially, circumferentially or even longitudinally or any other direction. The flow can be directed through concentric tubes, i.e. tube within a tube. Each material stream will have a dedicated inlet tube or, alternatively, plural streams may be inserted through a single inlet tube. Of course, if desired, each stream may be added through more than one inlet tube, in various combinations of like or different materials, quantities, feed rates, flow rates, concentrations, temperatures, etc.

One type of mixer that may be used in the process is a static mixer. Static mixers are well know in the art and are generally in the form of a series of repeating or random, interlocking plates and, or fins. Static mixers suitable for use in the process include the Chemineer SSC.75-4R-S (KMA 4 element ¾″) available from Chemineer Inc. P.O. Box 1123, Dayton, Ohio 45401 and the Koch SMX 4 element mixer (¾″ nominal) available from Koch-Glitsch LP Mass Transfer Sales and Engineering, 9525 Kenwood Road, Suite 16-246, Cincinnati, Ohio 45242. Another type of mixer is that may be used is a dynamic mixer. One type of dynamic mixer is a high shear mill, such as those available from IKA Works, Wilmington N.C. Further, if desired, static mixers or other inline mixers may be disposed in or with one or more of the inlet tubes or upstream of the confluence region. Additionally, surge tanks may be used to provide more constant flow for materials combined by the process described and claimed herein. Additionally or alternatively a Zanker plate may be utilized.

The choice of mixer is dependent upon the phase structure of the resultant composition. For example, for mixing some materials which are used to produce an isotropic composition, a static mixer is sufficient. For mixing other materials to produce a lamellar composition, one must use greater agitation to build the viscosity of the resultant composition. Therefore, a dynamic mixing system may be appropriate, such as a high shear mill. A dynamic mixing system as used herein is inclusive of the batch and continuous stir systems which use an impeller, jet mixing nozzle, a recirculating loop, gas percolation, rotating or fixed screen or similar means of agitation to combine materials therein.

The material streams will comprise a fluid, typically a liquid. Liquids are inclusive of suspensions, emulsions, slurries, aqueous and nonaqueous materials, pure materials, blends of materials, etc., all having a liquid state of matter. Any motive force or similar means for supplying the liquid streams, including pumps and servomotors may be used. As used herein motive force refers to any force used to provide energy which, in turn, is used to supply material streams to the confluence/mixing region and may include, without limitation, electric motors, gravity feeds, manual feeds, hydraulic feeds, pneumatic feeds, etc.

The material streams may be supplied from a hopper, tank, reservoir, pump, such as a positive displacement pump, or other supply or source to the pipe, or other supply devices, as are known in the art and provide the desired accuracy for dosing such materials.

The apparatus for providing the material streams may comprise a plurality of positive displacement pumps. Each pump may be driven by an associated motor, such as an AC motor or a servomotor. Each servomotor may be dedicated to a single pump or optionally may drive plural pumps. This arrangement eliminates the necessity of having flow control valves, flow meters and associated flow control feedback loops as are used in the prior art.

The processing system used and described herein may also employ an automatic control system. The control system may consist of any of a number of options available in the industry and known by one who is of ordinary skill in the art. One particularly suitable approach is to use a Programmable Logic Controller (PLC) such as Allen Bradley's ControlLogix® available from Rockwell Automation, Milwaukee, Wis. An operator interface may also be provided such as a personal computer with Wonderware (R) software.

The system described herein may also contain pumps or pressure regulating devices. These can be used to provide adequate and consistent pressure at any point in the process where it is required.

In one aspect, one or more of the processing system described herein may be employed or in conjunction with one or more additional processing systems and the products produced by employing multiple processing systems may be discharged into a common container, thereby forming for example, a product having multiple layers, phases, patterns etc. Such layers, phases and/or patterns may or may not mix in the container to form a homogeneous product. In one aspect, the processing system to manufacture a first phase of a product may be in a separate location from the processing system to produce a second or nth phase for filling the container with the final multi-phase composition, such as a dentifrice with a paste phase and a gel phase.

In one aspect, the processing system or multiple systems can be a coupled with a filling line to fill containers with a first phase, a second phase, combined phase and/or a multiphase composition. In one aspect, where the composition is intended to be combined with another composition to form a multiphase product it may be filled into containers in many ways. For example, one could fill containers by combining toothpaste-tube filling technology with a spinning stage design. Additionally, the present invention can be filled into containers by the method and apparatus as disclosed in U.S. Pat. No. 6,213,166 issued to Thibiant, et al. on Apr. 10, 2001. The method and apparatus allows two or more compositions to be filled in a spiral configuration into a single container using at least two nozzles to fill a container, which is placed on a rotating stage and spun as the composition is introduced into the container.

In one aspect, the components of the processing system described herein may be designed to be modular units that may be easily added to or deleted from a total process.

In another aspect, oral care compositions and products are produced by the processes disclosed herein. Typical components or ingredients of oral care compositions are described in the following paragraphs along with non-limiting examples. These ingredients include active agents and other orally acceptable carrier materials which are suitable for topical oral administration. By “compatible” is meant that the components of the composition are capable of being commingled without interaction in a manner which would substantially reduce composition stability and/or efficacy. Suitable active agents, carrier or excipient materials are well known in the art. Their selection will depend on desired activity, product form and secondary considerations like taste, cost, and shelf stability, etc. Their distribution among the base and other ingredient streams during manufacture will depend on the desired final composition and their physical and chemical properties. Any of the ingredients may be absent or present in more than one stream.

### Fluoride Source

It is common to have a fluoride compound present in dentifrices and other oral compositions in an amount sufficient to give a fluoride ion concentration in the composition of from about 0.0025% to about 5.0% by weight, preferably from about 0.005% to about 2.0% by weight to provide anticaries effectiveness. As discussed above, prevention of caries is essential for overall tooth health and integrity. A wide variety of fluoride ion-yielding materials can be employed as sources of soluble fluoride in the present compositions. Examples of suitable fluoride ion-yielding materials are found in U.S. Pat. No. 3,535,421 to Briner et al. and U.S. Pat. No. 3,678,154 to Widder et al. Representative fluoride ion sources include: stannous fluoride, sodium fluoride, potassium fluoride, amine fluoride, sodium monofluorophosphate, indium fluoride and many others.

### Antimicrobial Agent

The present compositions may include an antimicrobial agent, preferably a quaternary ammonium antimicrobial agent to provide bactericidal efficacy, i.e., effectiveness in killing, and/or altering metabolism, and/or suppressing the growth of, microorganisms which cause topically-treatable infections and diseases of the oral cavity, such as plaque, caries, gingivitis, and periodontal disease.

The antimicrobial quaternary ammonium compounds used in the compositions of the present invention include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethoylstearyl ammonium chloride, cetylpyridinium chloride, quaternized 5-amino-1,3 -bis(2-ethyl-hexyl)-5-methyl hexahydropyrimidine, benzalkonium chloride, benzethonium chloride and methyl benzethonium chloride are exemplary of typical quaternary ammonium antibacterial agents. Other compounds are bis[4-(R-amino)-1-pyridinium] alkanes as disclosed in U.S. Pat. No. 4,206,215, Jun. 3, 1980 to Bailey. The pyridinium compounds are the preferred quaternary ammonium compounds, particularly preferred being cetylpyridinium, or tetradecylpyridinium halide salts (i.e., chloride, bromide, fluoride and iodide). Most preferred is cetylpyridinium chloride. The quaternary ammonium antimicrobial agents are included in the present invention at levels of at least about 0.035%, preferably from about 0.045% to about 1.0%, more preferably from about 0.05% to about 0.10% by weight of the composition.

The present compositions may comprise a metal ion source that provides stannous ions, zinc ions, copper ions, or mixtures thereof as antimicrobial agent. The metal ion source can be a soluble or a sparingly soluble compound of stannous, zinc, or copper with inorganic or organic counter ions. Examples include the fluoride, chloride, chlorofluoride, acetate, hexafluorozirconate, sulfate, tartrate, gluconate, citrate, malate, glycinate, pyrophosphate, metaphosphate, oxalate, phosphate, carbonate salts and oxides of stannous, zinc, and copper.

Stannous, zinc and copper ions have been found to help in the reduction of gingivitis, plaque, sensitivity, and improved breath benefits. An effective amount is defined as from at least about 50 ppm to about 20,000 ppm metal ion of the total composition, preferably from about 500 ppm to about 15,000 ppm. More preferably, metal ions are present in an amount from about 3,000 ppm to about 13,000 ppm and even more preferably from about 5,000 ppm to about 10,000 ppm. This is the total amount of metal ions (stannous, zinc, copper and mixtures thereof) for delivery to the tooth surface.

Dentifrices containing stannous salts, particularly stannous fluoride and stannous chloride, are described in U.S. Pat. No. 5,004,597 to Majeti et al. Other descriptions of stannous salts are found in U.S. Pat. No. 5,578,293 issued to Prencipe et al. and in U.S. Pat. No. 5,281,410 issued to Lukacovic et al. In addition to the stannous ion source, other ingredients needed to stabilize the stannous may be included, such as the ingredients described in Majeti et al. and Prencipe et al.

The preferred stannous salts are stannous fluoride and stannous chloride dihydrate. Other suitable stannous salts include stannous acetate, stannous tartrate and sodium stannous citrate. Examples of suitable zinc ion sources are zinc oxide, zinc sulfate, zinc chloride, zinc citrate, zinc lactate, zinc gluconate, zinc malate, zinc tartrate, zinc carbonate, zinc phosphate, and other salts listed in U.S. Pat. No 4,022,880. Zinc citrate and zinc lactate are particularly preferred. Examples of suitable copper ion sources are listed in U.S. Pat. No. 5,534,243. The combined metal ion source(s) will be present in an amount of from about 0.05% to about 11%, by weight of the final composition. Preferably, the metal ion sources are present in an amount of from about 0.5 to about 7%, more preferably from about 1% to about 5%. Preferably, the stannous salts may be present in an amount of from about 0.1 to about 7%, more preferably from about 1% to about 5%, and most preferably from about 1.5% to about 3% by weight of the total composition. The amount of zinc or copper salts used in the present invention ranges from about 0.01 to about 5%, preferably from about 0.05 to about 4%, more preferably from about 0.1 to about 3.0%.

The present invention may also include other antimicrobial agents including non-cationic antimicrobial agents such as halogenated diphenyl ethers, phenolic compounds including phenol and its homologs, mono and poly-alkyl and aromatic halophenols, resorcinol and its derivatives, xylitol, bisphenolic compounds and halogenated salicylanilides, benzoic esters, and halogenated carbanilides. Also useful antimicrobials are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234 to Gieske et al. Examples of other antimicrobial agents include chlorhexidine, triclosan, triclosan monophosphate, and flavor oils such as thymol. Triclosan and other agents of this type are disclosed in Parran, Jr. et al., U.S. Pat. No. 5,015,466, and U.S. Pat. No. 4,894,220 to Nabi et al. These agents may be present at levels of from about 0.01% to about 1.5%, by weight of the dentifrice composition.

### Anticalculus Agent

The present compositions may optionally include an anticalculus agent, such as a pyrophosphate salt as a source of pyrophosphate ion. The pyrophosphate salts useful in the present compositions include the mono-, di- and tetraalkali metal pyrophosphate salts and mixtures thereof. Disodium dihydrogen pyrophosphate (Na₂H₂P₂O₇), sodium acid pyrophosphate, tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 0.025% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is a preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, Third Edition, Volume 17, Wiley-Interscience Publishers (1982).

Optional agents to be used in place of or in combination with the pyrophosphate salt include such known materials as longer chain (3 or more) polyphosphates including tripolyphosphate, tetrapolyphosphate and hexametaphosphate; synthetic anionic polymers, including polyacrylates and copolymers of maleic anhydride or acid and methyl vinyl ether (e.g., Gantrez), as described, for example, in U.S. Pat. No. 4,627,977, to Gaffar et al. as well as, e.g., polyamino propane sulfonic acid (AMPS), diphosphonates (e.g., EHDP; AHP), polypeptides (such as polyaspartic and polyglutamic acids), and mixtures thereof.

### Other Active Agents

Still another active agent that may be included in the present compositions is a tooth bleaching active selected from the group consisting of peroxides, perborates, percarbonates, peroxyacids, persulfates, and combinations thereof. Suitable peroxide compounds include hydrogen peroxide, urea peroxide, calcium peroxide, sodium peroxide, zinc peroxide and mixtures thereof. A preferred percarbonate is sodium percarbonate. Preferred persulfates are oxones.

Preferred peroxide sources for use in dentifrice formulations are calcium peroxide and urea peroxide. Hydrogen peroxide and urea peroxide are preferred for use in mouthrinse formulations. The following amounts represent the amount of peroxide raw material, although the peroxide source may contain ingredients other than the peroxide raw material. The present composition may contain from about 0.01% to about 30%, preferably from about 0.1% to about 10%, and more preferably from about 0.5% to about 5% of a peroxide source, by weight of the composition.

In addition to whitening, the peroxide also provides other benefits to the oral cavity. It has long been recognized that hydrogen peroxide and other peroxygen-compounds are effective in curative and/or prophylactic treatments with respect to caries, dental plaque, gingivitis, periodontitis, mouth odor, recurrent aphthous ulcers, denture irritations, orthodontic appliance lesions, postextraction and postperiodontal surgery, traumatic oral lesions and mucosal infections, herpetic stomatitis and the like. Peroxide-containing agents in the oral cavity exert a chemomechanical action generating thousands of tiny oxygen bubbles produced by interaction with tissue and salivary enzymes. The swishing action of a mouthrinse enhances this inherent chemomechanical action. Such action has been recommended for delivery of other agents into infected gingival crevices. Peroxide mouthrinses thus prevent colonization and multiplication of anaerobic bacteria known to be associated with periodontal disease.

Another optional active agent that may be added to the present compositions is a dentinal desensitizing agent to control hypersensitivity, such as salts of potassium, calcium, strontium and tin including nitrate, chloride, fluoride, phosphates, pyrophosphate, polyphosphate, citrate, oxalate and sulfate.

### Tooth Substantive Agent

The present invention may include a tooth substantive agent such as polymeric surface active agents (PMSA's), which are polyelectrolytes, more specifically anionic polymers. The PMSA's contain anionic groups, e.g., phosphate, phosphonate, carboxy, or mixtures thereof, and thus, have the capability to interact with cationic or positively charged entities. The “mineral” descriptor is intended to convey that the surface activity or substantivity of the polymer is toward mineral surfaces such as calcium phosphate minerals in teeth.

Tooth substantive agents provide many benefits including providing protection and resistance of teeth against erosion and wear derived from binding of calcium minerals in teeth (hydroxyapatite) and/or deposition on the tooth surface of a protective surface coating. Dental erosion is a permanent loss of tooth substance from the surface due to the action of chemicals, such as harsh abrasives and acids. The protective surface coating provides control of tooth surface characteristics including modification of surface hydrophilic and hydrophobic properties and resistance to acid attack. The tooth substantive agents may also provide desired surface conditioning effects including: 1) effective desorption of portions of undesirable adsorbed pellicle proteins, in particular those associated with tooth stain binding, calculus development and attraction of undesirable microbial species and 2) maintaining surface conditioning effects and control of pellicle film for extended periods following product use, including post brushing and throughout more extended periods. The effect of modifying the surface hydrophilic and hydrophobic properties can be measured in terms of changes in water contact angles, a relative decrease indicating a more hydrophilic surface and a relative increase indicating a more hydrophobic surface. Many of the tooth substantive agents also provide tartar control or antistain/whitening or surface conditioning activities, hence providing multiple clinical actions in improving overall health and structure of teeth as well as appearance and tactile impression of teeth. It is believed the tooth substantive agents provide a stain prevention benefit because of their reactivity or substantivity to mineral surfaces, resulting in desorption of portions of undesirable adsorbed pellicle proteins, in particular those associated with binding color bodies that stain teeth, calculus development and attraction of undesirable microbial species. The retention of these agents on teeth can also prevent stains from accruing due to disruption of binding sites of color bodies on tooth surfaces.

Suitable examples of PMSA tooth substantive agents are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); polycarboxylates and carboxy-substituted polymers; and mixtures thereof Suitable polymeric mineral surface active agents include the carboxy-substituted alcohol polymers described in U.S. Pat. Nos. 5,292,501; 5,213,789, 5,093,170; 5,009,882; and 4,939,284; all to Degenhardt et al. and the diphosphonate-derivatized polymers in U.S. Pat. No. 5,011,913 to Benedict et al; the synthetic anionic polymers including polyacrylates and copolymers of maleic anhydride or acid and methyl vinyl ether (e.g., Gantrez), as described, for example, in U.S. Pat. No. 4,627,977, to Gaffar et al. A preferred polymer is diphosphonate modified polyacrylic acid. Polymers with activity must have sufficient surface binding propensity to desorb pellicle proteins and remain affixed to enamel surfaces. For tooth surfaces, polymers with end or side chain phosphate or phosphonate functions are preferred although other polymers with mineral binding activity may prove effective depending upon adsorption affinity.

Additional examples of suitable phosphonate containing polymeric mineral surface active agents include the geminal diphosphonate polymers disclosed as anticalculus agents in U.S. Pat. No. 4,877,603 to Degenhardt et al; phosphonate group containing copolymers disclosed in U.S. Pat. No. 4,749,758 to Dursch et al. and in GB 1,290,724 (both assigned to Hoechst) suitable for use in detergent and cleaning compositions; and the copolymers and cotelomers disclosed as useful for applications including scale and corrosion inhibition, coatings, cements and ion-exchange resins in U.S. Pat. No. 5,980,776 to Zakikhani et al. and U.S. Pat. No. 6,071,434 to Davis et al. Additional polymers include the water-soluble copolymers of vinylphosphonic acid and acrylic acid and salts thereof disclosed in GB 1,290,724 wherein the copolymers contain from about 10% to about 90% by weight vinylphosphonic acid and from about 90% to about 10% by weight acrylic acid, more particularly wherein the copolymers have a weight ratio of vinylphosphonic acid to acrylic acid of 70% vinylphosphonic acid to 30% acrylic acid; 50% vinylphosphonic acid to 50% acrylic acid; or 30% vinylphosphonic acid to 70% acrylic acid. Other suitable polymers include the water soluble polymers disclosed by Zakikhani and Davis prepared by copolymerizing diphosphonate or polyphosphonate monomers having one or more unsaturated C═C bonds (e.g., vinylidene-1,1-diphosphonic acid and 2-(hydroxyphosphinyl)ethylidene-1,1-diphosphonic acid), with at least one further compound having unsaturated C═C bonds (e.g., acrylate and methacrylate monomers). Suitable polymers include the diphosphonate/acrylate polymers supplied by Rhodia under the designation ITC 1087 (Average MW 3000-60,000) and Polymer 1154 (Average MW 6000-55,000).

A preferred PMSA is a polyphosphate. A polyphosphate is generally understood to consist of two or more phosphate molecules arranged primarily in a linear configuration, although some cyclic derivatives may be present. Although pyrophosphates (n=2) are technically polyphosphates, the polyphosphates desired are those having around three or more phosphate groups so that surface adsorption at effective concentrations produces sufficient non-bound phosphate functions, which enhance the anionic surface charge as well as hydrophilic character of the surfaces. The inorganic polyphosphate salts desired include tripolyphosphate, tetrapolyphosphate and hexametaphosphate, among others. Polyphosphates larger than tetrapolyphosphate usually occur as amorphous glassy materials. Preferred in this invention are the linear polyphosphates having the formula:

XO(XPO₃)ₙX

wherein X is sodium, potassium or ammonium and n averages from about 3 to about 125. Preferred polyphosphates are those having n averaging from about 6 to about 21, such as those commercially known as Sodaphos (n≈6), Hexaphos (n≈13), and Glass H (n≈21) and manufactured by FMC Corporation and Astaris. These polyphosphates may be used alone or in combination. Polyphosphates are susceptible to hydrolysis in high water formulations at acid pH, particularly below pH 5. Thus it is preferred to use longer-chain polyphosphates, in particular Glass H with an average chain length of about 21. It is believed such longer-chain polyphosphates when undergoing hydrolysis produce shorter-chain polyphosphates which are still effective to deposit onto teeth and provide a stain preventive benefit. In addition to creating the surface modifying effects, the tooth substantive agent may also function to solubilize insoluble salts. For example, Glass H has been found to solubilize insoluble stannous salts. Thus, in compositions containing stannous fluoride for example, Glass H contributes to decreasing the stain promoting effect of stannous.

Other polyphosphorylated compounds may be used in addition to or instead of the polyphosphate, in particular polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof Preferred herein is phytic acid, also known as myo-inositol 1,2,3,4,5,6-hexakis (dihydrogen phosphate) or inositol hexaphosphoric acid, and its alkali metal, alkaline earth metal or ammonium salts. Herein, the term “phytate” includes phytic acid and its salts as well as the other polyphosphorylated inositol compounds.

Still other surface active organophosphate compounds useful as tooth substantive agents include phosphate mono-, di- or triesters represented by the following general structure wherein Z¹, Z², or Z³ may be identical or different, at least one being an organic moiety, preferably selected from linear or branched, alkyl or alkenyl group of from 6 to 22 carbon atoms, optionally substituted by one or more phosphate groups; alkoxylated alkyl or alkenyl, (poly)saccharide, polyol or polyether group.

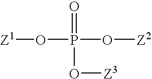

Some preferred agents include alkyl or alkenyl phosphate esters represented by the following structure:

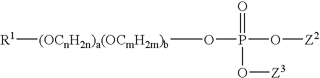

wherein R¹ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z² and Z³ may be identical or different, each represents hydrogen, alkali metal, ammonium, protonated alkyl amine or protonated functional alkyl amine such as an alkanolamine, or a R¹—(OCₙH₂ₙ)ₐ(OCₘH₂ₘ)_{b}— group. Examples of suitable agents include alkyl and alkyl (poly)alkoxy phosphates such as lauryl phosphate (tradenames MAP 230K and MAP 230T from Croda); PPG5 ceteareth-10 phosphate (available from Croda under the tradename Crodaphos SG); Laureth-1 phosphate (tradenames MAP L210 from Rhodia, Phosten HLP-1 from Nikkol Chemical or Sunmaep L from Sunjin); Laureth-3 phosphate (tradenames MAP L130 from Rhodia or Foamphos L-3 from Alzo or Emphiphos DF 1326 from Huntsman Chemical); Laureth-9 phosphate (tradename Foamphos L-9 from Alzo); Trilaureth-4 phosphate (tradenames Hostaphat KL 340D from Clariant or TLP-4 from Nikkol Chemical); C12-18 PEG 9 phosphate (tradename Crafol AP261 from Cognis); Sodium dilaureth-10 phosphate (tradename DLP-10 from Nikkol Chemical). Particularly preferred agents are polymeric, for example those containing repeating alkoxy groups as the polymeric portion, in particular 3 or more ethoxy, propoxy isopropoxy or butoxy groups.

Additional suitable polymeric organophosphate agents include dextran phosphate, polyglucoside phosphate, alkyl polyglucoside phosphate, polyglyceryl phosphate, alkyl polyglyceryl phosphate, polyether phosphates and alkoxylated polyol phosphates. Some specific examples are PEG phosphate, PPG phosphate, alkyl PPG phosphate, PEG/PPG phosphate, alkyl PEG/PPG phosphate, PEG/PPG/PEG phosphate, dipropylene glycol phosphate, PEG glyceryl phosphate, PBG (polybutylene glycol) phosphate, PEG cyclodextrin phosphate, PEG sorbitan phosphate, PEG alkyl sorbitan phosphate, and PEG methyl glucoside phosphate.

Suitable non-polymeric phosphates include alkyl mono glyceride phosphate, alkyl sorbitan phosphate, alkyl methyl glucoside phosphate, alkyl sucrose phosphates.

The amount of tooth substantive agent will typically be from about 0.1% to about 35% by weight of the total oral composition. In dentifrice formulations, the amount is preferably from about 2% to about 30%, more preferably from about 5% to about 25%, and most preferably from about 6% to about 20%. In mouthrinse compositions, the amount of tooth substantive agent is preferably from about 0.1% to 5% and more preferably from about 0.5% to about 3%.

### Chelating Agents

Another optional agent is a chelating agent, also called sequestrants, such as gluconic acid, tartaric acid, citric acid and pharmaceutically-acceptable salts thereof. Chelating agents are able to complex calcium found in the cell walls of the bacteria. Chelating agents can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus aid in preventing their adverse effects on the stability or appearance of products. Chelation of ions, such as iron or copper, helps retard oxidative deterioration of finished products.

Examples of suitable chelating agents are sodium or potassium gluconate and citrate; citric acid/alkali metal citrate combination; disodium tartrate; dipotassium tartrate; sodium potassium tartrate; sodium hydrogen tartrate; potassium hydrogen tartrate; sodium, potassium or ammonium polyphosphates and mixtures thereof. The chelating agent may be used from about 0.1% to about 2.5%, preferably from about 0.5% to about 2.5% and more preferably from about 1.0% to about 2.5%.

Still other chelating agents suitable for use in the present invention are the anionic polymeric polycarboxylates. Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Additional operative polymeric polycarboxylates are disclosed in U.S. Pat. No. 4,138,477 to Gaffar and U.S. Pat. No. 4,183,914 to Gaffar et al. and include copolymers of maleic anhydride with styrene, isobutylene or ethyl vinyl ether; polyacrylic, polyitaconic and polymaleic acids; and sulfoacrylic oligomers of MW as low as 1,000 available as Uniroyal ND-2.

### Surfactants

The present compositions will typically also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof. Preferred surfactants or surfactant mixtures are those that are compatible with the organophosphate agent and other actives in the composition in that the activities of these components are not compromised. Anionic surfactants, such as sodium alkyl sulfate and amphoteric surfactants, such as cocoamidopropyl betaine are preferred herein.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458. The present composition typically comprises an anionic surfactant at a level of from about 0.025% to about 9%, from about 0.05% to about 5% or from about 0.1% to about 1%.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate.

Zwitterionic or amphoteric surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577 to Polefka et al. Typical alkyl dimethyl betaines include decyl betaine or 2-(N-decyl-N,N-dimethylammonio) acetate, coco betaine or 2-(N-coco-N, N-dimethyl ammonio) acetate, myristyl betaine, palmityl betaine, lauryl betaine, cetyl betaine, cetyl betaine, stearyl betaine, etc. The amidobetaines are exemplified by cocoamidoethyl betaine, cocamidopropyl betaine (CADB), and lauramidopropyl betaine.

Cationic surfactants useful in the present invention include derivatives of quaternary ammonium compounds having one long alkyl chain containing from about 8 to 18 carbon atoms such as lauryl trimethylammonium chloride; cetyl pyridinium chloride; cetyl trimethylammonium bromide; coconut alkyltrimethylammonium nitrite; cetyl pyridinium fluoride; etc. Preferred compounds are the quaternary ammonium fluorides having detergent properties described in U.S. Pat. No. 3,535,421 to Briner et al. Certain cationic surfactants can also act as germicides in the compositions disclosed herein.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

### Abrasives

Dental abrasives useful in the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230 and DiGiulio, U.S. Pat. No. 3,862,307. Examples include the silica xerogels marketed under the trade name “Syloid” by the W.R. Grace & Company, Davison Chemical Division and precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in Wason, U.S. Pat. No. 4,340,583; and in commonly-assigned U.S. Pat. Nos. 5,603,920; 5,589,160; 5,658,553; 5,651,958; and 6,740,311. The silica abrasives described therein include various grades of silica such as standard or base silica and high-cleaning or high-polishing silica.

Mixtures of abrasives can be used such as mixtures of the various grades of Zeodent® silica abrasives listed above. The total amount of abrasive in dentifrice compositions of the subject invention typically range from about 6% to about 70% by weight; toothpastes preferably contain from about 10% to about 50% of abrasives. Dental solution, mouth spray, mouthwash and non-abrasive gel compositions of the subject invention typically contain little or no abrasive.

### Flavor System

The flavor system is typically added to oral care compositions, to provide a pleasant tasting composition and to effectively mask any unpleasant taste and sensations due to certain components of the composition such as antimicrobial actives or peroxide. Pleasant tasting compositions improve user compliance to prescribed or recommended use of oral care products. The present flavor system will comprise flavor components, in particular those that have been found to be relatively stable in the presence of usual oral care product actives, carrier materials or excipients. The combination of the selected flavoring components with sensate ingredients such as coolant(s) provides a high-impact refreshing sensation with a well-rounded flavor profile.

The flavor system may comprise flavor ingredients including but not limited to peppermint oil, corn mint oil, spearmint oil, oil of wintergreen, clove bud oil, cassia, sage, parsley oil, marjoram, lemon, lime, orange, cisjasmone, 2,5-dimethyl-4-hydroxy-3(2H)-furanone, 5-ethyl-3-hydroxy-4-methyl-2(5H)-furanone, vanillin, ethyl vanillin, anisaldehyde, 3,4-methylenedioxybenzaldehyde, 3,4-dimethoxybenzaldehyde, 4-hydroxybenzaldehyde, 2-methoxybenzaldehyde, benzaldehyde; cinnamaldehyde, hexyl cinnamaldehyde, alpha-methyl cinnamaldehyde, ortho-methoxy cinnamaldehyde, alpha-amyl cinnamaldehydepropenyl guaethol, heliotropine, 4-cis-heptenal, diacetyl, methyl-p-tert-butyl phenyl acetate, menthol, methyl salicylate, ethyl salicylate, 1-menthyl acetate, oxanone, alpha-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, alpha-terpineol, linalool, limonene, citral, maltol, ethyl maltol, anethole, dihydroanethole, carvone, menthone, β-damascenone, ionone, gamma decalactone, gamma nonalactone, gamma undecalactone and mixtures thereof. Generally suitable flavoring ingredients are those containing structural features and functional groups that are less prone to redox reactions. These include derivatives of flavor chemicals that are saturated or contain stable aromatic rings or ester groups. Also suitable are flavor chemicals that may undergo some oxidation or degradation without resulting in a significant change in the flavor character or profile. The flavor ingredients may be supplied in the composition as single or purified chemicals or by addition of natural oils or extracts that have preferably undergone a refining treatment to remove components that are relatively unstable and may degrade and alter the desired flavor profile, resulting in a less acceptable product from an organoleptic standpoint. Flavoring agents are generally used in the compositions at levels of from about 0.001% to about 5%, by weight of the composition.

The flavor system will typically include a sweetening agent. Suitable sweeteners include those well known in the art, including both natural and artificial sweeteners. Some suitable water-soluble sweeteners include monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexylen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used. A composition preferably contains from about 0.1% to about 10% of sweetener, by weight.

Suitable cooling agents or coolants include a wide variety of materials such as menthol and derivatives thereof Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds such as N-ethyl-p-menthan-3-carboxamide, known commercially as “WS-3”, and others in the series such as WS-5, WS-11, WS-14 and WS-30. An example of a synthetic carboxamide coolant that is structurally unrelated to menthol is N,2,3-trimethyl-2-isopropylbutanamide, known as “WS-23”. Additional suitable coolants include 3-1-menthoxypropane-1,2-diol known as TK-10, isopulegol (under the tradename Coolact P) and ρ-menthane-3,8-diol (under the tradename Coolact 38D) all available from Takasago; menthone glycerol acetal known as MGA; menthyl esthers such as menthyl acetate, menthyl acetoacetate, menthyl lactate known as Frescolat® supplied by Haarmann and Reimer, and monomenthyl succinate under the tradename Physcool from V. Mane. The terms menthol and menthyl as used herein include dextro- and levorotatory isomers of these compounds and racemic mixtures thereof. TK-10 is described in U.S. Pat. No. 4,459,425, Amano et al. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide.

In addition the flavor system may include salivating agents, hydration and moisturization agents, warming agents, and numbing agents. These agents are present in the compositions at a level of from about 0.001% to about 10%, preferably from about 0.1% to about 1%, by weight of the composition. Suitable salivating agents include Jambu® manufactured by Takasago and Optaflow® from Symrise. Examples of hydration agents include polyols such as erythritol. Suitable numbing agents include benzocaine, lidocaine, clove bud oil, and ethanol. Examples of warming agents include ethanol, capsicum and nicotinate esters, such as benzyl nicotinate. Use of agents with warming effects may of course alter the cooling effect of coolants and will need to be considered, particularly in optimizing the level of coolants.

### Miscellaneous Carrier Materials

Water employed in the preparation of commercially suitable oral compositions should preferably be of low ion content and free of organic impurities. Water may comprise up to about 99% by weight of the aqueous compositions herein. These amounts of water include the free water which is added plus that which is introduced with other materials, such as with sorbitol.

The present invention may also include an alkali metal bicarbonate salt, which may serve a number of functions including abrasive, deodorant, buffering and adjusting pH. Alkali metal bicarbonate salts are soluble in water and unless stabilized, tend to release carbon dioxide in an aqueous system. Sodium bicarbonate, also known as baking soda, is a commonly used bicarbonate salt. The present composition may contain from about 0.5% to about 30% by weight of an alkali metal bicarbonate salt.

The present compositions in the form of toothpastes, dentifrices and gels typically will contain some thickening material or binder to provide a desirable consistency. Preferred thickening agents are carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents are typically used in an amount from about 0.1% to about 15%, by weight.

Another optional component of the compositions desired herein is a humectant. The humectant serves to keep toothpaste compositions from hardening upon exposure to air and certain humectants can also impart desirable sweetness of flavor to toothpaste compositions. Suitable humectants for use in the invention include glycerin, sorbitol, polyethylene glycol, propylene glycol, and other edible polyhydric alcohols. The humectant generally comprises from about 0% to 70%, preferably from about 15% to 55%, by weight of the composition.

The pH of the present compositions may be adjusted through the use of buffering agents. Buffering agents, as used herein, refer to agents that can be used to adjust the pH of aqueous compositions such as mouthrinses and dental solutions preferably to a range of about pH 4.0 to about pH 8.0. Buffering agents include sodium bicarbonate, monosodium phosphate, trisodium phosphate, sodium hydroxide, sodium carbonate, sodium acid pyrophosphate, citric acid, and sodium citrate and are typically included at a level of from about 0.5% to about 10% by weight.

Poloxamers may be employed in the present compositions. A poloxamer is classified as a nonionic surfactant and may also function as an emulsifying agent, binder, stabilizer, and other related functions. Poloxamers are difunctional block-polymers terminating in primary hydroxyl groups with molecular weights ranging from 1,000 to above 15,000. Poloxamers are sold under the tradename of Pluronics and Pluraflo by BASF including Poloxamer 407 and Pluraflo L4370.

Other emulsifying agents that may be used include polymeric emulsifiers such as the Pemulen® series available from B.F. Goodrich, and which are predominantly high molecular weight polyacrylic acid polymers useful as emulsifiers for hydrophobic substances.

Titanium dioxide may also be added to the present compositions as coloring or opacifying agent typically at a level of from about 0.25% to about 5% by weight.

Other optional agents that may be used in the present compositions include dimethicone copolyols selected from alkyl- and alkoxy-dimethicone copolyols, such as C12 to C20 alkyl dimethicone copolyols and mixtures thereof, as aid in providing positive tooth feel benefits. Highly preferred is cetyl dimethicone copolyol marketed under the trade name Abil EM90. The dimethicone copolyol is generally present from about 0.01% to about 25%, preferably from about 0.1% to about 5 by weight.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope.

Examples of dentifrice compositions that may be may be produced by the processes disclosed herein are shown in Table 2. The amount shown in weight % for each material is the amount in the final product after combining the base and other material streams comprised of the dentifrice ingredients.

TABLE 2
Dentifrice Compositions

| Ingredient | 1A | 1B | 1C | 1D | 1E | 1F | 1G |
|---|---|---|---|---|---|---|---|
| Water | 38.51 | 23.26 | 23.26 | 8.0 | 8.95 | 13.7 | — |
| Glycerin | — | — | — | 9.00 | — | 7.750 | 36.944 |
| Sorbitol 70% soln. | 24.21 | 33.80 | 32.80 | 41.0 | 60.0 | 24.91 | — |
| Polyethylene Glycol 300 | — | 3.720 | 3.720 | 3.00 | — | 6.00 | 7.000 |
| Propylene Glycol | — | — | — | — | — | — | 7.000 |
| Silica Z-109 | — | — | 7.667 | — | — | — | 12.500 |
| Silica Z-119 | 21.00 | 17.00 | 9.333 | 17.0 | 15.0 | 31.0 | 12.500 |
| Tetrasodium Pyrophosphate | — | 1.128 | 1.128 | 3.850 | — | 5.045 | — |
| Disodium Pyrophosphate | — | 1.344 | 1.344 | 1.0 | — | — | — |
| Tetrapotassium Pyrophosphate | — | 3.159 | 3.159 | — | — | — | — |
| Sodium Polyphosphate | — | — | — | — | — | — | 13.000 |
| Sodium Fluoride | 0.32 | 0.321 | 0.321 | 0.243 | 0.243 | 0.243 | — |
| Stannous Fluoride | — | — | — | — | — | — | 0.454 |
| Triclosan/PEG Premix | — | 0.560 | 0.560 | — | — | — | — |
| Monosodium Phosphate | — | — | — | — | 0.419 | — | — |
| Trisodium Phosphate | 0.37 | — | — | — | 1.10 | — | 1.100 |
| Sodium Carbonate | — | — | — | — | — | 0.500 | — |
| Sodium Bicarbonate | — | — | — | — | — | 1.500 | — |
| Sodium Gluconate | — | — | — | — | — | — | 0.652 |
| Zinc Lactate Dihydrate | — | — | — | — | — | — | 2.500 |
| Xanthan Gum | — | 0.500 | 0.500 | 0.475 | — | — | 0.250 |
| Carbomer 956 | 0.30 | 0.300 | 0.300 | 0.300 | 0.300 | — | — |
| Na Carboxymethylcellulose | 1.10 | 0.700 | 0.700 | — | 0.750 | 0.750 | — |
| Carrageenan | — | — | — | — | — | — | 0.600 |
| Sodium Saccharin | 0.20 | 0.200 | 0.200 | 0.40 | 0.130 | 0.350 | 0.500 |
| Sodium Lauryl Sulfate 28% Soln | 2.00 | — | — | 2.0 | 2.0 | 5.0 | 3.400 |
| Poloxamer | — | — | — | — | — | 1.25 | — |

The following examples further illustrate dentifrice compositions made according to the processes disclosed herein Components and number of the streams are shown. The various streams are separately prepared and mixed to produce the final product. The amount shown in weight % for each material is the amount in the final product after combining the base stream(s) and other ingredient streams.

Example 1

| | |
|---|---|
| Number of Base Streams | 1 |
| Number of Aqueous Streams | 1 |
| Number of Organic Streams | 1 |
| Number of Non-aqueous Streams | 0 |
| Base Stream (s) | |
| Water | 38.506% |
| Sorbitol | 26.057% |
| Silica - Base | 21.000% |
| NaF | 0.321% |
| CMC | 1.100% |
| Carbomer | 0.300% |
| Sodium Lauryl Sulfate Soln | 0.150% |
| Saccharin | 0.200% |
| Trisodium Phosphate | 0.366% |
| Aqueous Stream (s) | |
| SLSS | 6.850% |
| Sorbitol | 3.550% |
| Titanium Dioxide | 0.250% |
| Saccharin | 0.200% |
| Organic Stream (s) | |
| Flavor | 1.150% |
| | 100.000% |

Example 2

| | |
|---|---|
| Number of Base Streams | 1 |
| Number of Aqueous Streams | 1 |
| Number of Organic Streams | 1 |
| Number of Non-aqueous Streams | 0 |
| Base Stream (s) | |
| Water | 38.506% |
| Sorbitol | 26.057% |
| Silica - Base | 21.000% |
| NaF | 0.321% |
| CMC | 1.100% |
| Carbomer | 0.300% |
| Sodium Lauryl Sulfate Soln | 0.150% |
| Saccharin | 0.200% |
| Trisodium Phosphate | 0.366% |
| Aqueous Stream (s) | |
| SLSS | 6.850% |
| Sorbitol | 3.785% |
| Saccharin | 0.200% |
| Blue Pigment | 0.015% |
| Flavor | 1.150% |
| | 100.000% |

Example 3

| | |
|---|---|
| Number of Base Streams | 1 |
| Number of Aqueous Streams | 1 |
| Number of Organic Streams | 1 |
| Number of Non-aqueous Streams | 0 |
| Base Stream (s) | |
| Water | 23.261% |
| Sorbitol | 35.807% |
| Silica - Base | 9.333% |
| Silica - High Cleaning | 7.667% |
| NaF | 0.321% |
| CMC | 0.700% |
| Xanthan Gum | 0.500% |
| Carbomer | 0.300% |
| Sodium Lauryl Sulfate Soln | 0.150% |
| Saccharin | 0.200% |
| Disodium Pyrophosphate | 1.344% |
| Tetrapotassium Pyrophosphate (60%) | 3.159% |
| Polyethylene Glycol | 4.000% |
| Triclosan | 0.280% |
| Aqueous Stream (s) | |
| SLSS | 7.000% |
| Sorbitol | 4.303% |
| Titanium Dioxide | 0.250% |
| Saccharin | 0.250% |
| BFGs Blue | 0.125% |
| Organic Stream (s) | |
| Flavor | 1.050% |
| | 100.000% |

Example 4

| | |
|---|---|
| Number of Base Streams | 2 |
| Number of Aqueous Streams | 3 |
| Number of Organic Streams | 1 |
| Number of Non-aqueous Streams | 1 |
| Base Stream (s) | |
| Sorbitol | 53.056% |
| Silica - Base | 15.000% |
| Silica - High Cleaning | 7.000% |
| NaF | 0.243% |
| CMC | 0.300% |
| Xanthan Gum | 0.300% |
| Saccharin | 0.200% |
| Disodium Pyrophosphate | 0.250% |
| Sodium Hydroxide 50% Solution | 0.410% |
| Aqueous Stream (s) | |
| SLSS | 4.000% |
| Sorbitol | 3.059% |
| Saccharin | 0.300% |
| Disodium Pyrophosphate | 3.920% |
| Blue Color Solution | 0.200% |
| Water | 8.000% |
| Carbomer | 0.300% |
| Sodium Hydroxide 50% Solution | 1.112% |
| Organic Stream (s) | |
| Flavor | 0.950% |
| Non-aqueous Stream (s) | |
| Glycerin | 0.500% |
| Propylene Glycol | 0.250% |
| Polyethylene Glycol | 0.250% |
| Xanthan Gum | 0.400% |
| | 100.000% |

Example 5

| | |
|---|---|
| Number of Base Streams | 1 |
| Number of Aqueous Streams | 3 |
| Number of Organic Streams | 1 |
| Number of Non-aqueous Streams | 2 |
| Base Stream (s) | |
| Water | 36.000% |
| Sorbitol | 13.803% |
| Silica - Base | 15.000% |
| NaF | 0.243% |
| Xanthan Gum | 0.250% |
| Sodium Lauryl Sulfate Soln | 0.150% |
| Saccharin | 0.200% |
| Disodium Pyrophosphate | 0.250% |
| Sodium Hydroxide 50% Solution | 0.410% |
| Aqueous Stream (s) | |
| SLSS | 6.850% |
| Sorbitol | 7.302% |
| Titanium Dioxide | 0.250% |
| Saccharin | 0.200% |
| Disodium Pyrophosphate | 2.530% |
| Blue Color Solution | 0.200% |
| Water | 9.000% |
| Carbomer | 0.400% |
| Sodium Hydroxide 50% Solution | 1.112% |
| Organic Stream (s) | |
| Flavor | 0.950% |
| Non-aqueous Stream (s) | |
| Glycerin | 1.750% |
| Propylene Glycol | 0.875% |
| Polyethylene Glycol | 0.875% |
| CMC | 1.400% |
| | 100.000% |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A semi-continuous process for manufacturing oral care compositions comprising:
a. preparing one or more base formulations comprising from about 40% to about 99% by weight of the oral care composition, to form one or more base stream(s) for further processing;
b. storing said one or more base formulations;
c. preparing one or more additive formulations selected from predominantly aqueous formulation(s) comprising greater than 50% water, essentially non-aqueous formulation(s) comprising less than 5% water and organic formulation comprising organic components, to form additive streams for combining with said one or more base stream(s);
d. storing said one or more additive formulations;
e. transferring said one or more base stream(s) and said one or more additive stream(s) into a mixing region;
f. contacting and mixing in said mixing region said one or more base stream(s) and said one or more additive stream(s) to form an oral care composition; and
g. transferring said oral care composition to a filling line for transfer to one or a plurality of containers.

2. The process of claim 1, comprising a simultaneous injection system for transferring said one or more base stream(s) and said one or more additive stream(s) into a mixing region.
